(19) United States
(12) Patent Application Publication
(10) Pub. No.: US 2024/0290465 A1
(43) Pub. Date: Aug. 29, 2024

(54) LIFESTYLE IMPROVEMENT SYSTEM, PORTABLE TERMINAL, AND CONTROL METHOD

(71) Applicant: Panasonic Intellectual Property Management Co., Ltd., Osaka (JP)

(72) Inventors: Hiroaki OMAYU, Osaka (JP); Tomoki OGAWA, Osaka (JP); Yuka NAKAMURA, Osaka (JP); Masafumi SATO, Osaka (JP); Kohsuke YAMAMOTO, Osaka (JP)

(21) Appl. No.: 18/572,639

(22) PCT Filed: Jun. 30, 2022
(86) PCT No.: PCT/JP2022/026346

§ 371 Date: Dec. 20, 2023

Related U.S. Application Data
(60) Provisional application No. 63/216,914, filed on Jun. 30, 2021.

(51) Int. Cl. G16H 20/70

(52) U.S. Cl. CPC G16H 20/70

(57) ABSTRACT

A lifestyle improvement system includes: an obtainer that obtains a plurality of first stages of behavior change by evaluating a stage of behavior change of each of a plurality of first users; a classifier that classifies the plurality of first users into a plurality of groups according to the plurality of first stages of behavior change, grouping users evaluated to be in similar stages of behavior change together; an extractor that obtains one or more first behaviors of each of one or more second users belonging to a first group among the plurality of groups, and extracts, from among the one or more first behaviors obtained, a second behavior that affects transition to a higher stage of behavior change; and a notifier that notifies one or more third users currently belonging to the first group among the one or more second users with behavior information indicating the second behavior.

### CROSS-REFERENCE OF RELATED APPLICATIONS

This application is the U.S. National Phase under 35 U.S.C. § 371 of International Patent Application No. PCT/JP2022/026346, filed on Jun. 30, 2022, which in turn claims the benefit of U.S. Provisional patent application Ser. No. 63/216,914, filed on Jun. 30, 2021, the entire disclosures of which Applications are incorporated by reference herein.

### TECHNICAL FIELD

The present disclosure relates to a lifestyle improvement system, a portable terminal, and a control method.

### BACKGROUND ART

Patent Literature (PTL) 1 discloses a dietary guidance support device that estimates the phase of behavior change (stage of behavior change) of a subject of dietary guidance based on a behavior change model, and provides dietary guidance support to the subject according to their phase of behavior change.

### CITATION LIST

### Patent Literature

[PTL 1] Japanese Unexamined Patent Application Publication No. 2016-091184

### SUMMARY OF INVENTION

### Technical Problem

The present disclosure provides a lifestyle improvement system, etc., that can effectively raise the stage of behavior change of a first user who receives support for lifestyle improvement and a second user who supports the lifestyle improvement of the first user.

### Solution to Problem

A lifestyle improvement system according to one aspect of the present disclosure includes: an obtainer that obtains a plurality of first stages of behavior change by evaluating a stage of behavior change of each of a plurality of first users; a classifier that classifies the plurality of first users into a plurality of groups according to the plurality of first stages of behavior change, grouping users evaluated to be in similar stages of behavior change together; an extractor that obtains one or more first behaviors of each of one or more second users belonging to a first group among the plurality of groups, and extracts, from among the one or more first behaviors obtained, a second behavior that affects transition to a higher stage of behavior change; and a notifier that notifies one or more third users currently belonging to the first group among the one or more second users with behavior information indicating the second behavior.

A portable terminal according to one aspect of the present disclosure is connected to a server over a network. The server includes: an obtainer that obtains a plurality of first stages of behavior change by evaluating a stage of behavior change of each of a plurality of first users; a classifier that classifies the plurality of first users into a plurality of groups according to the plurality of first stages of behavior change, grouping users evaluated to be in similar stages of behavior change together; an extractor that obtains one or more first behaviors of each of one or more second users belonging to a first group among the plurality of groups, and extracts, from among the one or more first behaviors obtained, a second behavior that affects transition to a higher stage of behavior change; and a notifier that notifies one or more third users currently belonging to the first group among the one or more second users with behavior information indicating the second behavior. The portable terminal includes: a receiver that receives the behavior information; and a presenter that presents the second behavior based on the behavior information received.

A control method according to one aspect of the present disclosure is a control method of a lifestyle improvement system including one or more processors and a presenter, and includes, executed by the one or more processors: obtaining a plurality of first stages of behavior change by evaluating a stage of behavior change of each of a plurality of first users; obtaining one or more first behaviors of each of one or more second users belonging to a first group among a plurality of groups into which the plurality of first users have been classified, the plurality of first users being classified according to the plurality of first stages of behavior change, grouping users evaluated to be in similar stages of behavior change together; and notifying one or more third users currently belonging to the first group among the one or more second users with behavior information indicating a second behavior that affects transition to a higher stage of behavior change from among the one or more first behaviors obtained.

These general or specific aspects may be implemented as a device/apparatus, an integrated circuit, a computer program, or a computer-readable recording medium such as a CD-ROM, or any combination thereof.

### Advantageous Effects of Invention

The lifestyle improvement system, etc., according to the present disclosure can effectively raise the stage of behavior change of a user who receives support for lifestyle improvement and a user who supports the lifestyle improvement of the previously mentioned user.

### BRIEF DESCRIPTION OF DRAWINGS

FIG. 1 is a graph for illustrating changes in disease structure.

FIG. 2 is a graph illustrating percentages of medical expenditures for different lifestyle-related diseases.

FIG. 3 is a graph illustrating the relationship between blood pressure categories and stroke incidence.

FIG. 4 illustrates factors that reduce blood pressure.

FIG. 5 illustrates factors that prevent people from continuing to improve their lifestyle habits.

FIG. 6 is a diagram illustrating a model of behavior change.

FIG. 7 illustrates one example of a functional block diagram of a lifestyle improvement system according to an embodiment.

FIG. 8 illustrates the configuration of a portable terminal in detail.

FIG. 9 is a sequence diagram illustrating a first example of operations performed by a lifestyle improvement system for notifying support information.

FIG. 10 is a sequence diagram illustrating a second example of operations performed by a lifestyle improvement system for notifying support information.

FIG. 11 is a flowchart illustrating one example of operations related to input of food consumption log entries in the lifestyle improvement system.

FIG. 12 is a flowchart illustrating a first example of a process of inputting a food consumption log.

FIG. 13 is a flowchart illustrating a second example of a process of inputting a food consumption log.

FIG. 14 is a flowchart illustrating a third example of a process of inputting a food consumption log.

FIG. 15 is a flowchart illustrating a fourth example of a process of inputting a food consumption log.

### DESCRIPTION OF EMBODIMENTS

### Background

The background of the invention will be explained.

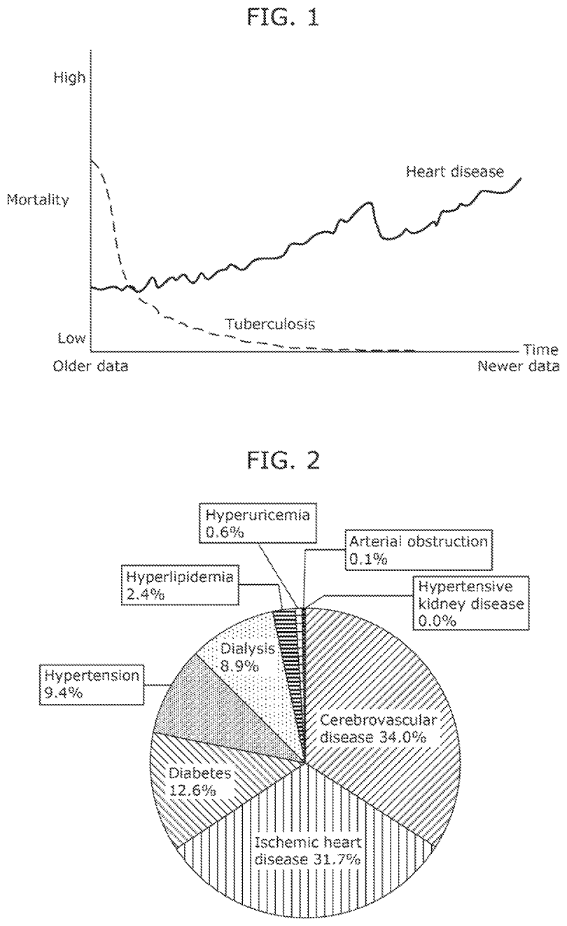

FIG. 1 is a graph for illustrating changes in disease structure.

As illustrated in FIG. 1, the structure of diseases affecting the Japanese population has shifted from infectious diseases to lifestyle-related diseases as sanitary conditions improved in the postwar period. For example, mortality from infectious diseases such as tuberculosis and pneumonia has been decreasing over time, while mortality from lifestyle-related diseases such as heart disease has been increasing. Lifestyle-related diseases are difficult to cure, so the cost of treatment is an ongoing problem. Additionally, in lifestyle-related diseases, the intensity of the patient's subjective symptoms does not correspond to the necessity of medical intervention.

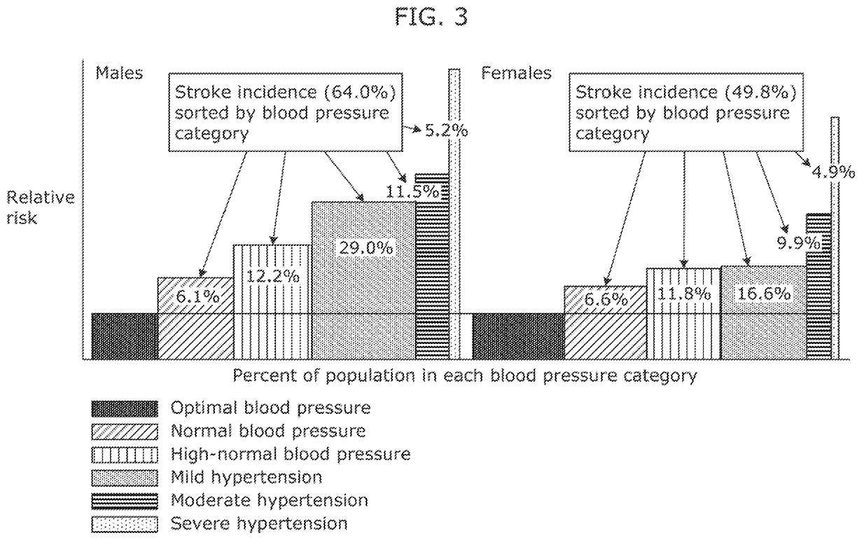

FIG. 2 is a graph illustrating percentages of medical expenditures for different lifestyle-related diseases. FIG. 3 is a graph illustrating the relationship between blood pressure categories and stroke incidence.

As illustrated in FIG. 2, about 75% of the medical expenditures for lifestyle-related diseases are spent on diseases in which high blood pressure is the main cause (cerebrovascular disease, ischemic heart disease, and hypertension). Multiple studies have also found that the risk of disease increases with higher blood pressure. For example, as illustrated in FIG. 3, the risk of stroke increases with higher blood pressure.

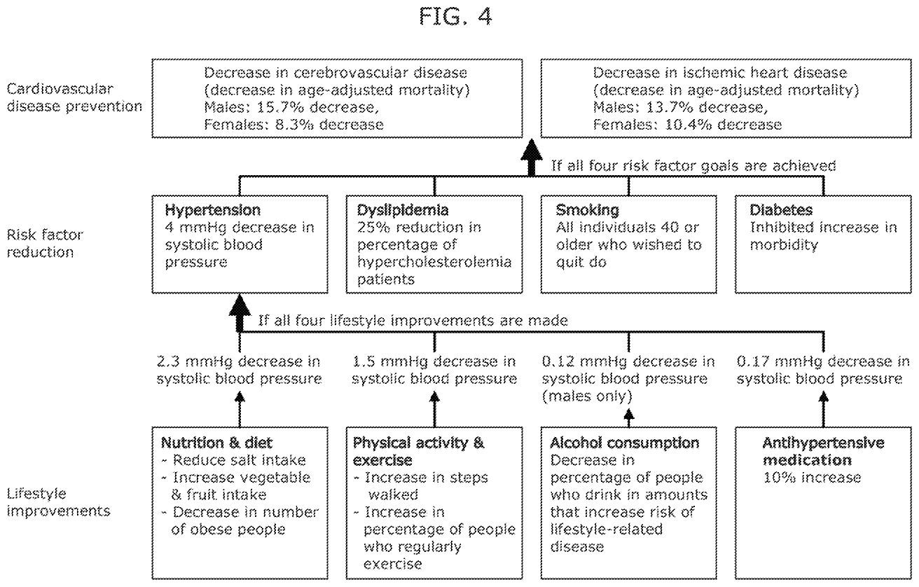

FIG. 4 illustrates factors that reduce blood pressure. FIG. 4 is a diagram based on the Guidelines for the Management of Hypertension 2019.

As illustrated in FIG. 4, four measures are considered important to reduce blood pressure: (1) nutrition and diet, (2) physical activity and exercise, (3) alcohol consumption, and (4) antihypertensive medication rates. Of these, (1) nutrition and diet, (2) physical activity and exercise, and (3) alcohol consumption are not medical measures, but measures related to improving one's everyday lifestyle.

In other words, improving one's everyday lifestyle is important for reducing lifestyle-related diseases.

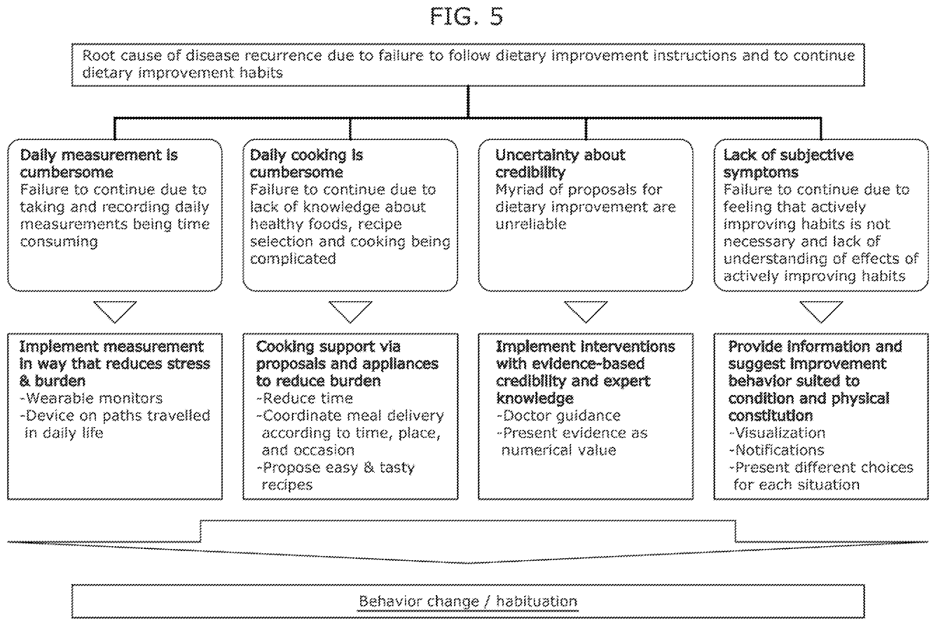

FIG. 5 illustrates factors that prevent people from continuing to improve their lifestyle habits.

In order to improve lifestyle habits in patients with lifestyle-related diseases, it is necessary to habituate behaviors that lead to good health on a patient by patient basis. If a person is able to habituate behaviors that lead to good health, it will help promote and maintain their health.

However, it is difficult to sustain lifestyle improvement behavior without following lifestyle (for example, diet) improvement instructions. Four conceivable factors for this difficulty are, for example, (1) daily measurement is cumbersome, (2) daily cooking is cumbersome, (3) uncertainty about the credibility of the information, and (4) lack of subjective symptoms. The four factors can be improved to habituate lifestyle improvement behaviors (behavior change). For example, (1) could be improved by implementing measurement in a way that reduces stress and burden. For example, (2) could be improved by implementing meal proposals that reduce the burden of cooking and implementing cooking support via home appliances. For example, (3) could be improved by implementing interventions with evidence-based credibility and expert knowledge. For example, (4) could be improved by providing information and suggesting improvement behavior suited to the condition and physical constitution of the individual.

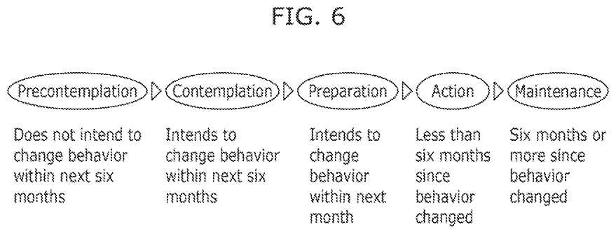

FIG. 6 is a diagram illustrating a model of behavior change.

It is common for individuals to change their behavior in gradual stages. As illustrated in FIG. 6, individuals are considered to progress through five stages of behavior change: precontemplation, contemplation, preparation, action, and maintenance. These five stages make up the model of behavior change, which is being researched and practiced on various health-related behaviors. In order to advance even one stage of behavior change, it is first necessary to know which stage the individual is in and to work with them according to the stage. The stages of behavior change are evaluated from low to high in the order of precontemplation, contemplation, preparation, action, and maintenance. In other words, the stages of behavior change of precontemplation, contemplation, preparation, action, and maintenance are listed in ascending order in terms of their evaluation, such that the contemplation stage is evaluated higher than the precontemplation stage, the preparation stage is evaluated higher than the contemplation stage, the action stage is evaluated higher than the preparation stage, and the maintenance stage is evaluated higher than the action stage. Thus, an individual's stage of behavior change can be said to be an indicator of their degree of willingness to change their daily behavior to transition from a first state to a second, improved state.

For example, the Ministry of Health, Labour and Welfare website (https://www.e-healthnet.mhlw.go.jp/information/exercise/s-07-001.html) discloses a model of behavior change and stage-appropriate approaches, which are described below.

Approaches for the precontemplation stage include, for example, raising awareness, learning about the benefits of physical activity, emotional experiences, realizing that the status quo needs to change, re-evaluating one's environment, and realizing how one's behavior impacts their surroundings.

Approaches for the contemplation stage include, for example, re-evaluation of oneself, and presenting physical inactivity as harmful and physical activity as beneficial.

Approaches for the preparation stage include, for example, personal determination and having confidence in one's ability to perform physical activity well and declaring to others that one is ready to begin physical activity.

Approaches for the action and maintenance stages include, for example, behavior replacement, replacing unhealthy behaviors with healthy ones (for example, using physical activity instead of alcohol to cope with stress), supportive relationships, using the support of others to continue physical activity, reinforcement management, giving ‘rewards’ for continuing physical activity, stimulus control, and creating an environment conducive to physical activity.

### Blood Pressure Monitor

In recent years, wearable cuffless blood pressure monitors have been developed to automatically measure blood pressure at regular intervals. Cuffless blood pressure monitors are approved as medical devices in some countries or regions.

Unlike conventional blood pressure monitors, cuffless blood pressure monitors are devices that are worn, for example, on the wrist or upper arm, and enable frequent and automatic blood pressure measurements at predetermined times without the user noticing, such as at 30-minute intervals, or when predetermined conditions are met (for example, when the user is at rest and there is little movement).

Conventionally, a blood pressure monitor with a cuff called Ambulatory Blood Pressure Monitoring (ABPM) has been used to obtain frequent blood pressure readings (for example, hourly blood pressure readings), but the periodic tightening of the cuff places stress on the subject's body. For this reason, frequent blood pressure values are rarely measured except in medical facilities when truly needed.

On the other hand, rather than using a cuff, cuffless blood pressure monitors obtain blood pressure readings by estimating blood pressure based on, for example, pulse waves obtained from an optical sensor, with little or no tightening of the body measurement position (wrist, upper arm, etc.). Thus, blood pressure can be measured frequently without placing stress on the subject's body as with ABPM.

### Smartphone Apps for Supporting Improvement Behavior

In recent years, health management apps have been developed that keep a record of the user's physical condition, such as keeping a food consumption log (content of daily meals), an exercise log (content of daily exercise), and a weight log, to visualize the user's health condition for dieting purposes. In such a health management app, for example, data recorded daily, such as a food consumption log, an exercise log, and physical condition, is checked by a nutritionist, and advice from the nutritionist is provided according to the user's diet goals. This allows the user to follow the advice of their nutritionist to improve their eating habits and exercise routines. Moreover, the app allows the user to browse a large number of cooking recipes and cook using recipes that follow the advice of their nutritionist.

In addition to manual input by the user, meal contents may be input in this app by citing cooking recipes in the app or by taking a photo of the meal and having the AI in the app predict the meal contents from the photo and using the prediction results. Moreover, meal contents may be input in this app by searching for store names and product names and selecting store names and product names if they are affiliated with the app. Reducing the time and complexity of entering meal contents is known to encourage continued use of the app.

### Problem 1

To improve hypertension or some other disease, when a user who receives support for lifestyle improvement (hereinafter “supported user”) and a user who supports the lifestyle improvement of the supported user (hereinafter “supporting user”) work together to improve the lifestyle (for example, eating habits) of the supported user, the following problems must be addressed. Here, the supported user and the supporting user are partners, such as a married couple. For example, if the supported user and the supporting user are in different stages of behavior change, trouble may occur between the supported user and the supporting user, and the supported user may not be able to make good progress in their efforts to improve their lifestyle and consequently may not engage in lifestyle improvement behavior. Note that a supported user refers to a user (patient) who has a disease such as hypertension and needs to improve their lifestyle in order to improve the disease, and a supporting user refers to a user who helps the supported user improve their lifestyle, for example, by cooking the supported user's meals.

The inventors found that this problem is based on, for example, the relationship between and the magnitudes of the stages of behavior change, as shown below.

(1) When the Supported User's Stage of Behavior Change is Higher than the Supporting User's Stage of Behavior Change

The supported user may not be conscious of improving their lifestyle and may not listen to the supporting user's recommendations to eat a low-sodium diet, making it difficult for both parties to discuss improvement behaviors. As a result, the supporting user loses motivation to support the supported user in improving their lifestyle.

Even if the supporting user prepares and serves a low-sodium meal, the supported user may have a preconceived notion that the meal will not taste good before eating it, and will not want to eat it. The supporting user loses motivation to support the supported user in improving their lifestyle when the supported user does not eat the low-sodium meal they cooked for them.

Even if the supporting user prepares a low-sodium meal, the supported user may complain that the meal does not taste good and will not eat much. The supporting user may also gradually lose motivation if the supported user does not make an effort to improve their lifestyle.

Even if the supporting user prepares a low-sodium meal, the supported user may be not satisfied and eat a heavily seasoned meal when the supporting user is not around (for example, by eating out). The supporting user loses motivation to support the supported user when they find out that the supported user is eating as they please behind their back.

(2) When the Supported User's Stage of Behavior Change is Lower than the Supporting User's Stage of Behavior Change

The supporting user does may not have the awareness to cook a low-sodium meal for the supported user, so the supported user performs the improvement behavior alone.

The supporting user may have a preconceived notion that low-sodium food does not taste good and may not cook low-sodium meals. As a result, the supported user may have no choice but to improve their diet outside of the home, limiting their means of improvement.

Suppose the supporting user wants to cook a low-sodium meal, but low-sodium meals often include fish dishes, which require the supporting user to be highly skilled at cooking.

Even if the supporting user initially cooperates and prepares low-sodium meals, the supporting user may not want to continue eating them themselves, so they may stop preparing low-sodium meals. As a result, the supported user may have no choice but to improve their diet outside of the home, limiting their means of improvement.

### (3) When the Supported User and the Supporting User are at the Same Stage of Behavior

The supported user and the supporting user may both have a preconceived notion that low-sodium meals do not taste good before eating them, and will not want to eat them. As a result, the stage of behavior change of the supported user is not raised and health improvement does not progress.

Even if the supported user and the supporting user prepare a low-sodium meal, they may complain that the meal does not taste good and not eat much. As a result, both the supported user and the supporting user will start to prepare low-sodium meals less frequently, impeding progression of their health improvement.

### Problem 2

There is a problem that the supported user or the supporting user alone will not be able to continue trying to improve the lifestyle of the supported user.

Lifestyle improvement for the treatment of lifestyle-related diseases is known to be difficult to sustain alone. On the other hand, it is known that creating a community with members who are in the same health condition and have the same goals, and supporting each other within the community, can increase motivation and help with sustaining lifestyle improvement more so than doing it alone. However, one problem with this approach is that effectively raising one's stage of behavior change is not taken into consideration.

In order to overcome Problem 2 above, the inventors of the present application discovered a lifestyle improvement system and the like that can effectively raise the stage of behavior change of at least the lower of the first user, who receives support for lifestyle improvement, and the second user, who supports the lifestyle improvement of the first user.

A lifestyle improvement system according to a first aspect of the present disclosure includes: an obtainer that obtains a plurality of first stages of behavior change by evaluating a stage of behavior change of each of a plurality of first users; a classifier that classifies the plurality of first users into a plurality of groups according to the plurality of first stages of behavior change, grouping users evaluated to be in similar stages of behavior change together; an extractor that obtains one or more first behaviors of each of one or more second users belonging to a first group among the plurality of groups, and extracts, from among the one or more first behaviors obtained, a second behavior that affects transition to a higher stage of behavior change; and a notifier that notifies one or more third users currently belonging to the first group among the one or more second users with behavior information indicating the second behavior.

With this, since one or more third users currently belonging to the first group are notified with the second behavior that affects the transition to a higher stage of behavior change among one or more first behaviors of each of one or more second users belonging to the first group, it is possible to raise the stage of behavior change of the third user to a higher stage of behavior change.

A lifestyle improvement system according to a second aspect of the present disclosure is the lifestyle improvement system according to the first aspect, wherein the one or more first behaviors are behaviors of the one or more third users.

With this, since the second behavior is extracted from among the behaviors of one or more third users currently belonging to the first group, it is possible to notify behavior information indicating the behavior of a user classified in the same group as the one or more third users subject to be notified. Accordingly, there is potential to more effectively raise the stage of behavior change of the one or more third users to a higher stage of behavior change.

A lifestyle improvement system according to a third aspect of the present disclosure is the lifestyle improvement system according to the first aspect, wherein the one or more first behaviors are behaviors that influenced one or more fourth users currently belonging to a second group evaluated at a higher stage of behavior change relative to the first group to transition from the first group to the second group.

With this, since a behavior of one or more fourth users who belonged to the first group which affected their transition from the first group to the second group is extracted as the second behavior, it is possible to extract a behavior having a proven track record of affecting group transitions as the second behavior. In other words, there is potential to more effectively raise the stage of behavior change of the one or more third users to a higher stage of behavior change.

A lifestyle improvement system according to a fourth aspect of the present disclosure is the lifestyle improvement system according any one of the first through third aspects, wherein the plurality of first users include a plurality of supported users who receive support for lifestyle improvement and a plurality of supporting users who support the lifestyle improvement of the plurality of supported users.

A lifestyle improvement system according to a fifth aspect of the present disclosure is the lifestyle improvement system according to the first aspect, wherein the first stage of behavior change of each of the plurality of supported users is determined at predetermined intervals according to at least one of: a record of daily activity of the supported user; a viewing history on a first terminal possessed by the supported user of an app for managing a health condition of the supported user; biometric information of the supported user; or an amount of exercise by the supported user, and the first stage of behavior change of each of the plurality of supporting users is determined at predetermined intervals according to a viewing history on a second terminal possessed by the supporting user of an app for managing a health condition of the supporting user, and a supporting skill of the supporting user.

With this, since a first stage of behavior change appropriate for the behavior and state of the supported user and a second stage of behavior change appropriate for the behavior and state of the supporting user are determined, it is possible to notify one or more users currently belonging to the first group of the second behavior that can effectively raise the stage of behavior change of the low-evaluated user.

A lifestyle improvement system according to a sixth aspect of the present disclosure is the lifestyle improvement system according to any one of the first through fifth aspects, wherein the stage of behavior change is an indicator of a degree of willingness to change daily behavior to transition from a first state to a second state improved relative to the first state.

This makes it possible to determine a stage of behavior change according to the degree of the user's willingness to change their daily behavior.

A portable terminal according to a seventh aspect of the present disclosure is connected to a server over a network. The server includes: an obtainer that obtains a plurality of first stages of behavior change by evaluating a stage of behavior change of each of a plurality of first users; a classifier that classifies the plurality of first users into a plurality of groups according to the plurality of first stages of behavior change, grouping users evaluated to be in similar stages of behavior change together; an extractor that obtains one or more first behaviors of each of one or more second users belonging to a first group among the plurality of groups, and extracts, from among the one or more first behaviors obtained, a second behavior that affects transition to a higher stage of behavior change; and a notifier that notifies one or more third users currently belonging to the first group among the one or more second users with behavior information indicating the second behavior. The portable terminal includes: a receiver that receives the behavior information; and a presenter that presents the second behavior based on the behavior information received.

With this, since the portable terminal notifies a third user currently belonging to the first group with the second behavior that affects the transition to a higher stage of behavior change among one or more first behaviors of each of one or more second users belonging to the first group, it is possible to raise the stage of behavior change of the third user to a higher stage of behavior change.

A control method according to an eighth aspect of the present disclosure is a control method of a lifestyle improvement system including one or more processors and a presenter, and includes, executed by the one or more processors: obtaining a plurality of first stages of behavior change by evaluating a stage of behavior change of each of a plurality of first users; obtaining one or more first behaviors of each of one or more second users belonging to a first group among a plurality of groups into which the plurality of first users have been classified, the plurality of first users being classified according to the plurality of first stages of behavior change, grouping users evaluated to be in similar stages of behavior change together; and notifying one or more third users currently belonging to the first group among the one or more second users with behavior information indicating a second behavior that affects transition to a higher stage of behavior change from among the one or more first behaviors obtained.

With this, since one or more third users currently belonging to the first group are notified with the second behavior that affects the transition to a higher stage of behavior change among one or more first behaviors of each of one or more second users belonging to the first group, it is possible to raise the stage of behavior change of the third user to a higher stage of behavior change.

These general or specific aspects may be implemented as a device/apparatus, an integrated circuit, a computer program, or a computer-readable recording medium such as a CD-ROM, or any combination thereof.

Hereinafter, embodiments will be described in detail with reference to the drawings, but unnecessarily detailed descriptions may be omitted. For example, detailed descriptions of well-known matters or descriptions of elements that are substantially the same as previously described elements may be omitted. This is to avoid redundancy and provide easily read descriptions for those skilled in the art.

Note that the inventors have provided the appended drawings and following description for the purpose of facilitating sufficient understanding of the present disclosure by those skilled in the art. Accordingly, the appended drawings and following description are not intended to limit the scope of the claims.

### EMBODIMENT

### Configuration

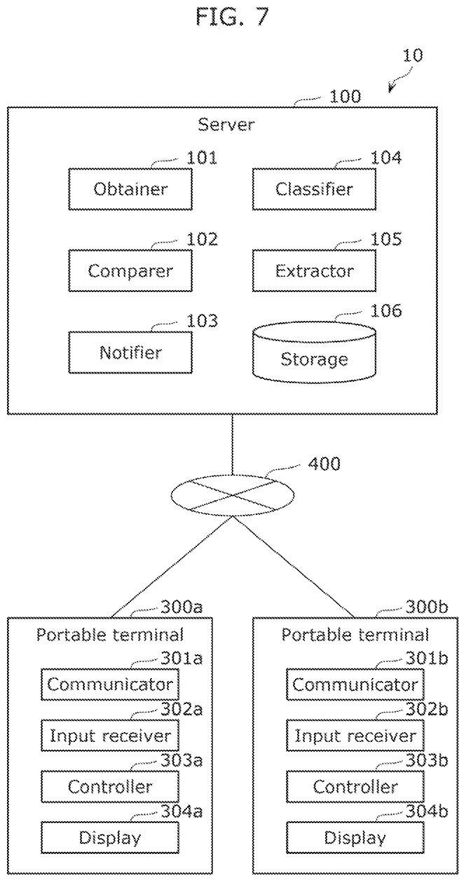

FIG. 7 illustrates one example of a functional block diagram of a lifestyle improvement system according to an embodiment.

As illustrated in FIG. 7, lifestyle improvement system 10 includes server 100 and portable terminals 300a and 300b. Server 100 and portable terminals 300a and 300b are communicably connected via network 400. Network 400 may be a general-purpose line, such as the Internet, or a dedicated line. Portable terminal 300a is a terminal carried by the first user. Portable terminal 300b is a terminal carried by the second user. The first user is a supported user who receives help in improving their lifestyle. The second user is a supporting user who supports the first user in improving their lifestyle. Here, “support” is, for example, support for the improvement of the first user's eating habits, and is the cooking of the first user's meals. Support is not limited to cooking the first user's meals, and also includes support for improving overall lifestyle habits not limited to eating habits, by encouraging the first user to lead a well-regulated lifestyle and exercise, and working on it together with the first user.

### Server

Server 100 is a cloud server that provides the first and second users with information for supporting the improvement of the first user's lifestyle. Server 100 includes obtainer 101, comparer 102, notifier 103, classifier 104, extractor 105, and storage 106. Server 100 is not required to include classifier 104 and extractor 105. Server 100 is also not required to include comparer 102. Server 100 includes a processor and memory. The functions of server 100 may be realized by a processor executing a program stored in memory.

Obtainer 101 is a communication interface (IF) that obtains various information from portable terminals 300a and 300b by communicating with portable terminals 300a and 300b via network 400. Obtainer 101 may obtain food consumption information of the first user from portable terminal 300a. The food consumption information is information about the first user's food consumption generated from information entered by the first user via a health management app on portable terminal 300a. The health management app manages the health condition of the first user and runs on portable terminals 300a and 300b. The food consumption information includes, for example, the frequency with which the first user takes pictures of meals they eat, pictures of meals, the frequency of eating out, the amount or frequency of snacking, and an evaluation of how the meals they eat taste. The food consumption information may include the dishes of a meal. Obtainer 101 may also obtain the dishes of the meal as food consumption information by estimating the dishes from a photo of the meal. Obtainer 101 may obtain the nutritional composition of a meal by estimating it from the dishes of the meal. Nutritional composition includes the amount of energy and salt content of the meal. In addition to food consumption information, obtainer 101 may also obtain lifestyle information indicating the first user's daily activities. In addition to food consumption information regarding the first user's meals, the lifestyle information may include information regarding the first user's basic activities essential for living, such as sleeping and defecating.

Obtainer 101 may also obtain app information about the health management app on portable terminal 300a. The app information includes the frequency with which the first user views the health management app on portable terminal 300a and the amount of instructional content geared for the first user that the first user has viewed in the health management app.

Obtainer 101 may also obtain the first user's biometric information, which is obtained from a measurement device not illustrated. The biometric information may include the first user's weight, blood pressure, pulse rate, body movements, number of steps, etc. The measurement device may be a scale or a blood pressure monitor, and may be an activity meter that includes a heart rate sensor, a triaxial accelerometer, and a triaxial angular rate sensor. The measurement device may transmit the obtained biometric information to server 100 via portable terminal 300a by communicating with portable terminal 300a, and it may transmit the obtained biometric information to server 100 by communicating with network 400.

Obtainer 101 may evaluate the stage of behavior change of the first user based on the food consumption information, app information, and biometric information obtained from portable terminal 300a, and take the evaluation result as the first stage of behavior change. Obtainer 101 may sequentially obtain food consumption information, app information, and biometric information from portable terminal 300a at predetermined intervals, and determine the first stage of behavior change at each predetermined interval according to at least one of the sequentially obtained food consumption information, app information, or biometric information. More specifically, obtainer 101 may determine the first stage of behavior change such that a higher stage of behavior is adopted in correlation with an increase in the frequency with which the first user takes pictures of meals they eat, a decrease in at least one of the amount of energy or salt in the nutritional composition of a meal in the food consumption information, a decrease in frequency of eating out, a decrease in at least one of the amount or frequency of snacks eaten, an increase in at least one of frequency of opening the health management app or the amount of instructional content viewed in the health management app, and an increase in the frequency with which the first user measures at least one of their weight or blood pressure.

Obtainer 101 may also obtain an evaluation of the first user's behavior by the second user or by the first user themselves, and take these evaluations into consideration when determining the first stage of behavior change. For example, obtainer 101 may determine the first stage of behavior change to be the precontemplation stage based on an evaluation of unwillingness to eat meals prepared with recipes designed to improve health. For example, obtainer 101 may determine the first stage of behavior change to be the contemplation stage based on an evaluation that the first user realizes that meals prepared with recipes designed to improve health are healthy (i.e., they show interest) but lack the knowledge of how or why they are healthy. For example, obtainer 101 may determine the first stage of behavior change to be the preparation stage based on an evaluation that the first user is willing to eat meals prepared with recipes designed to improve health but is not acting on it (i.e., is not eating such meals). For example, obtainer 101 may determine the first stage of behavior change to be the action stage based on an evaluation or a track record that the first user is eating meals prepared with recipes designed to improve health but is not satisfactorily feeling the health benefits. For example, obtainer 101 may determine the first stage of behavior change to be the maintenance stage based on an evaluation or track record that the first user continues to eat meals prepared with recipes designed to improve health, snacks less than before, and has a daily energy and salt intake below a certain value.

Obtainer 101 may also determine the first stage of behavior change according to the status of the registration of food consumption information and the evaluation thereof. For example, obtainer 101 may determine the first stage of behavior change to be the precontemplation stage when no food consumption log photos are entered or no biometric information is recorded. For example, obtainer 101 may determine the first stage of behavior change to be the contemplation stage based on an evaluation that the first user understands that keeping a food consumption log will provide meal content estimations or guidance, but does not understand why it is necessary for him or her to do so. For example, obtainer 101 may determine the first stage of behavior change to be the preparation stage based on an evaluation that the first user is willing to keep a food consumption log but is not acting on it (i.e., has not kept a food consumption log). For example, obtainer 101 may determine the first stage of behavior change to be the action stage based on an evaluation that the first user has kept a food consumption log but is not satisfactorily feeling the health benefits. For example, obtainer 101 may determine the first stage of behavior change to be the maintenance stage based on an evaluation that the first user is continuing to keep a food consumption log, has changed their meal content, or has reduced their estimated daily energy or salt intake to a certain value, or that the biometric information has changed.

Obtainer 101 may also determine the first stage of behavior change according to the status of the registration of the first user's biometric information and the evaluation thereof. For example, obtainer 101 may determine the first stage of behavior change to be the precontemplation stage based on an evaluation of unwillingness to record biometric information. For example, obtainer 101 may determine the first stage of behavior change to be the contemplation stage based on an evaluation that the first user understands (was told by a doctor) that recording biometric information is necessary, but does not understand why it is necessary for him or her to do so. For example, obtainer 101 may determine the first stage of behavior change to be the preparation stage based on an evaluation that the first user is willing to record biometric information but is not acting on it (i.e., has not recorded biometric information). For example, obtainer 101 may determine the first stage of behavior change to be the action stage based on an evaluation that the first user has recorded biometric information but is not satisfactorily feeling the health benefits. For example, obtainer 101 may determine the first stage of behavior change to be the maintenance stage based on an evaluation that the first user continues to record biometric information, or that the first user feels a change in biometric information, or that the first user understands the relationship (their body's unique relationship) between diet and biometric information.

Note that the timing for obtaining food consumption information, app information, and biometric information by obtainer 101 is not limited to the above examples; it is sufficient so long as obtainer 101 can determine the first stage of behavior change at predetermined intervals according to at least one of the food consumption information, the app information, or the biometric information. Accordingly, obtainer 101 may obtain the first stage of behavior change by determining the first stage of behavior change.

Obtainer 101 may also obtain the first stage of behavior change determined by portable terminal 300a at predetermined time intervals from portable terminal 300a. Obtainer 101 may also obtain the first stage of behavior change determined by an external device not illustrated at predetermined time intervals from the external device. The first stage of behavior change is determined based on the food consumption information, the app information, and the biometric information generated by portable terminal 300a. Note that portable terminal 300a may be any terminal used by the first user, and may be two or more terminals, not just one.

Obtainer 101 may obtain app information about the health management app on portable terminal 300b. The app information includes the amount of time the second user views recipes designed to improve health (for example, low-sodium recipes) in the health management app on portable terminal 300b, the frequency that the second user inputs information indicating that the second user cooked a meal using a recipe designed to improve health on that day, the frequency that the second user views the health management app, the amount of instructional content geared for the second user viewed by the second user in the health management app. Obtainer 101 may also estimate the amount and rate of decrease in the use of seasonings, including salt, included in recipes cooked based on input history over a predetermined period of information in the health management app indicating that a meal using a recipe designed to improve health was prepared. Obtainer 101 may estimate the cooking skill level of the second user based on the app information.

Obtainer 101 may evaluate the stage of behavior change of the second user based on the app information obtained from portable terminal 300b, and take the evaluation result as the second stage of behavior change. Obtainer 101 may determine the second stage of behavior change at predetermined intervals according to the app information from portable terminal 300b. More specifically, obtainer 101 may determine the second stage of behavior change such that a higher stage of behavior is adopted in correlation with an increase in the amount of time the second user views recipes designed to improve health in the health management app, an increase in the frequency that the second user inputs information indicating that the second user cooked a meal using a recipe designed to improve health on that day, an increase in at least one of frequency of opening the health management app or the amount of instructional content viewed in the health management app, a decrease in the amount and rate of decrease in the use of seasonings, including salt, included in recipes cooked, and an increase in cooking skill level.

Obtainer 101 may also obtain an evaluation of the second user's behavior by the first user or by the second user themselves, and take these evaluations into consideration when determining the second stage of behavior change. For example, obtainer 101 may determine the second stage of behavior change to be the precontemplation stage based on an evaluation of unwillingness to cook meals prepared with recipes designed to improve health. For example, obtainer 101 may determine the second stage of behavior change to be the contemplation stage based on an evaluation that the second user realizes that meals prepared with recipes designed to improve health are healthy (i.e., they show interest) but has a preconceived notion that such meals do not taste good or are time-consuming. For example, obtainer 101 may determine the second stage of behavior change to be the preparation stage based on an evaluation that the second user is willing to cook meals prepared with recipes designed to improve health but is not acting on it (i.e., is not cooking such meals). For example, obtainer 101 may determine the second stage of behavior change to be the action stage based on an evaluation or a track record that the second user is cooking meals prepared with recipes designed to improve health but is not satisfactorily feeling the health benefits. For example, obtainer 101 may determine the second stage of behavior change to be the maintenance stage based on an evaluation or a track record that the second user is continuing to cook meals prepared with recipes designed to improve health, does not mind reducing salt in their usual cooking, or thinks such meals taste good.

Note that the timing for obtaining app information by obtainer 101 is not limited to the above examples; it is sufficient so long as obtainer 101 can determine the second stage of behavior change at predetermined intervals according to the app information. Accordingly, obtainer 101 may obtain the second stage of behavior change by determining the second stage of behavior change. Obtainer 101 may also obtain the second stage of behavior change determined by portable terminal 300b from portable terminal 300b. Obtainer 101 may also obtain the second stage of behavior change determined by an external device not illustrated from the external device. The second stage of behavior change is determined based on the app information generated by portable terminal 300b. Note that portable terminal 300b may be any terminal used by the second user, and may be two or more terminals, not just one.

Obtainer 101 may obtain a plurality of first stages of behavior change by evaluating the stage of behavior change of each of a plurality of first users. Obtainer 101 may obtain a plurality of second stages of behavior change by evaluating the stage of behavior change of each of a plurality of second users. A plurality of first users refers to a plurality of supported users, and a plurality of second users refers to a plurality of supporting users. Stated differently, obtainer 101 may obtain a plurality of stages of behavior change by evaluating each of a plurality of users including a plurality of supported users and a plurality of supporting users.

Comparer 102 compares the first stage of behavior change with the second stage of behavior change to identify a difference between the first stage of behavior change and the second stage of behavior change. For example, comparer 102 may determine that there is a difference if the first stage of behavior change and the second stage of behavior change are different from each other, and determine that there is no difference if they are the same as each other. If the first stage of behavior change and the second stage of behavior change are different from each other, comparer 102 may determine how many stages there are between them.

If comparer 102 determines that there is a difference, notifier 103 notifies a low-evaluated user, which is a user whose stage of behavior change is evaluated low among the first user and the second user, with first support information corresponding to the stage of behavior change of the low-evaluated user and to the difference. The first support information is information presented to the low-evaluated user for raising the stage of behavior change of the low-evaluated user. Notifier 103 determines, as the first support information to notify the low-evaluated user with, first support information that is associated with a combination of the first stage of behavior change and the second stage of behavior change from among a plurality of instances of the first support information stored in storage 106.

If comparer 102 determines that there is a difference, notifier 103 further notifies a high-evaluated user, which is a user whose stage of behavior change is evaluated high among the first user and the second user, with second support information corresponding to the stage of behavior change of the high-evaluated user and to the difference. The second support information is information presented to the high-evaluated user for maintaining or raising the stage of behavior change of the high-evaluated user. Notifier 103 determines, as the second support information to notify the high-evaluated user with, second support information that is associated with a combination of the first stage of behavior change and the second stage of behavior change from among a plurality of instances of the second support information stored in storage 106. For example, the first support information includes at least one of advice, encouragement, a suggestion, or behavior information indicating a lifestyle improvement behavior of the high-evaluated user, each of which is for raising the stage of behavior change of the low-evaluated user. For example, the second support information includes at least one of advice, encouragement, a suggestion, or behavior information indicating an improvement behavior of the low-evaluated user, to maintain or raise the stage of behavior change of the high-evaluated user.

Classifier 104 classifies a plurality of users including a plurality of supported users and a plurality of supporting users into a plurality of groups according to the plurality of stages of behavior change obtained by evaluating each of the plurality of users, such that users evaluated to be in similar stages of behavior change are classified into the same group. Classifier 104 may classify a plurality of first users into a plurality of groups such that first users evaluated to be in similar stages of behavior change are classified into the same group. Classifier 104 may classify a plurality of second users into a plurality of groups such that second users evaluated to be in similar stages of behavior change are classified into the same group. The plurality of groups may be, for example, five groups corresponding to the five stages of behavior change in which the first user can be evaluated. The plurality of groups may be, for example, fewer than five groups, with users classified into two or more stages of behavior change in a single group.

Extractor 105 obtains one or more first behaviors of each of one or more users belonging to a first group among the plurality of groups, and extracts a second behavior that affects the transition to a higher stage of behavior change from among the obtained one or more first behaviors. Notifier 103 may then notify one or more users currently belonging to the first group with the second behavior.

Here, the one or more first behaviors may be behaviors of one or more users currently belonging to the first group. Stated differently, extractor 105 extracts the second behavior from among one or more first behaviors of one or more users currently belonging to the first group. The second behavior in such cases is a behavior that is presumed to be more likely to influence the transition to the group associated with the next higher stage of behavior change (for example, a second group) relative to the first group.

The one or more first behaviors may be behaviors that influenced one or more users currently belonging to a second group evaluated at a higher stage of behavior change relative to the first group to transition from the first group to the second group. Stated differently, extractor 105 may extract the second behavior from one or more first behaviors of one or more users who previously belonged to the first group and currently belong to the second group associated with a higher stage of behavior change relative to the first group. In such cases, the second behavior is the behavior that had affected the transition to the second group.

Storage 106 stores a plurality of instances of the first support information. Each instance of the first support information is associated with a plurality of combinations of a plurality of stages of behavior change (for example, the five stages of precontemplation, contemplation, preparation, action, and maintenance) in which the first user is evaluatable and a plurality of stages of behavior change (for example, the five stages of precontemplation, contemplation, preparation, action, and maintenance) in which the second user is evaluatable. Storage 106 also stores a plurality of instances of the second support information. Each instance of the second support information is associated with a plurality of combinations of a plurality of stages of behavior change (for example, the five stages of precontemplation, contemplation, preparation, action, and maintenance) in which the first user is evaluatable and a plurality of stages of behavior change (for example, the five stages of precontemplation, contemplation, preparation, action, and maintenance) in which the second user is evaluatable.

### Specific Example of First Support Information

For example, if the second user's stage of behavior change is the precontemplation stage and the first user's stage of behavior change is the precontemplation stage or higher, the first support information may be instructional content that is provided via a health app and has the effect of improving food literacy and motivation, such as instructional content on the dangers of neglecting high blood pressure, that a small lifestyle change can be effective, and that low-sodium meals can be delivered as meal kits or boxed lunches.

For example, if the second user's stage of behavior change is the contemplation stage and the first user's stage of behavior change is the contemplation stage or higher, the first support information may be information (a message) suggesting the second user to eat a meal kit or low-sodium boxed lunch before preparing and eating a low-sodium meal. Having the first and second users actually eat a meal kit or low-sodium boxed lunch is done in the hope that it will dispel preconceived notions that low-sodium meals do not taste good.

In such cases, the first support information may be instructional content that improves the second user's cooking skills, such as how to handle fish.

In such cases, if the first user is found to have been on a healthy diet with less calories (energy) and salt than usual when eating out, the first support information may be the content of the first user's diet or a message that the first user is making an effort to improve their eating habits.

For example, if the second user's stage of behavior change is the preparation stage and the first user's stage of behavior change is the preparation stage or higher, the first support information may be information (a message) suggesting to eat a meal kit or low-sodium boxed lunch before preparing and eating a low-sodium meal. Having the first and second users actually eat a meal kit or low-sodium boxed lunch is done in the hope that it will dispel preconceived notions that low-sodium meals do not taste good.

In such cases, the first support information may be instructional content for improving cooking skills, such as how to handle fish.

In such cases, if the first user is found to have been on a healthy diet with less calories (energy) and salt than usual when eating out, the first support information may be the content of the first user's diet or a message that the first user is making an effort to improve their eating habits.

In such cases, for example, when the second user once ate a low-sodium dish and evaluated that it did not taste good, the first support information may be information (a message) suggesting the first and second users to eat a meal kit or a low-sodium boxed lunch.

In such cases, for example, when the second user once ate a low-sodium dish and evaluated that it did not taste good, the first support information may be information (a message) suggesting the first and second users to use a low-sodium recipe for side dishes instead of main dishes.

### Examples of the Relationship Between Difference and Support Information

The first example is when the first user's stage of behavior change is the contemplation stage and the second user's stage of behavior change is the preparation stage. The first user is aware of a health issue but does not know how to address it, so their stage of behavior change is evaluated to be the contemplation stage. The second user knows how to address the health issue but is not taking actions to improve their health, so their stage of behavior change is evaluated to be the preparation stage.

In the first example, since the first user is at a lower stage of behavior change than the second user, the first user is the target of the notification of the first support information, and since the second user does not yet have a concrete track record (but may have started activity partially), support of correct knowledge and information is tailored to the first user. For example, the first support information includes a message informing the first user about the health risks of leaving hypertension untreated. For example, the first support information includes a message informing the first user that the recommended salt intake is 6 grams per day. For example, the first support information includes a message informing the first user about meal issues and improvements the first user can make, based on the first user's food consumption log information.

The second example is when the first user's stage of behavior change is the contemplation stage and the second user's stage of behavior change is the action stage. The first user is aware of a health issue but does not know how to address it, so their stage of behavior change is evaluated to be the contemplation stage. The second user is taking actions to improve their health accordingly, but since there is a possibility they may quit, their stage of behavior change is evaluated to be the action stage.

In the second example, since the first user is at a lower stage of behavior change than the second user, the first user is the target of the notification of the first support information, and since the second user has reached the action stage, in addition to support of correct knowledge and information tailored to the first user, the second user's implementation behavior (effort) is conveyed to the first user. In addition, it is possible to provide learning opportunities (advice) through shopping and other activities together, which can be expected to lead to motivation to improve lifestyle habits since it provides correct knowledge and rewards the second user for their ingenuity and efforts (gratitude). For example, the first support information may include the following information in addition to the same message as in the first example. For example, the first support information includes information for sharing the menu or recipe information that the second user is working on with the first user. For example, the first support information includes information encouraging the first user to enter their impressions of the menu or recipe. For example, the second support information includes information provided to the second user for sharing these impressions entered by the first user with the second user. For example, the first support information includes information for sharing shopping information for the menu or recipe with the first user or for requesting the sharing from the second user. For example, the first support information includes a message for the first user suggesting the first user to cook together with the second user.

The third example is when the first user's stage of behavior change is the preparation stage and the second user's stage of behavior change is the contemplation stage. The first user knows how to address the health issue but is not taking actions to improve their health, so their stage of behavior change is evaluated to be the preparation stage. The second user is aware of a health issue but does not know how to address it, so their stage of behavior change is evaluated to be the contemplation stage.

In the third example, since the second user is at a lower stage of behavior change than the first user, the second user is the target of the notification of the first support information, and since the first user does not yet have a concrete track record (but may have started activity partially), support of correct knowledge and information is tailored to the second user. For example, the first support information includes a message informing the second user about the health risks of leaving hypertension untreated. For example, the first support information includes a message informing the second user that simply changing the seasoning to a low-sodium alternative is effective (i.e., that a small change is all that is needed).

The fourth example is when the first user's stage of behavior change is the action stage and the second user's stage of behavior change is the contemplation stage. The first user is taking actions to improve their health accordingly, but since there is a possibility they may quit, their stage of behavior change is evaluated to be the action stage. The second user is aware of a health issue but does not know how to address it, so their stage of behavior change is evaluated to be the contemplation stage.

In the fourth example, since the second user is at a lower stage of behavior change than the first user, the second user is the target of the notification of the first support information, and since the first user has reached the action stage, in addition to support of correct knowledge and information tailored to the second user, the first user's implementation behavior (effort) is conveyed to the second user. In addition, sharing the first user's changes with the second user can be expected to lead to motivation for the second user, as the one who does the cooking, to support the first user in improving their lifestyle. For example, the first support information may include the following information in addition to the same message as in the third example. For example, the first support information includes information for sharing with the second user changes in the first user's food consumption log (salt intake), blood pressure, and weight.

### Portable Terminals

Portable terminals 300a and 300b are terminals such as smartphones or tablets possessed by a user.

Portable terminal 300a includes communicator 301a, input receiver 302a, controller 303a, and display 304a. Portable terminal 300b includes communicator 301b, input receiver 302b, controller 303b, and display 304b. Communicator 301a, input receiver 302a, controller 303a, and display 304a included in portable terminal 300a are the same as communicator 301b, input receiver 302b, controller 303b, and display 304b included in portable terminal 300b, respectively. Therefore, the configuration of portable terminal 300a will be described. Portable terminals 300a and 300b include a processor and memory. The functions of portable terminals 300a and 300b may be realized by the processor executing a program stored in the memory. Hereinafter, where there is no need for distinction, portable terminals 300a and 300b may be referred to in the singular simply as portable terminal 300.

Communicator 301a exchanges information with server 100 via network 400. Communicator 301a receives various support information (for example, first support information, second support information, etc.) from server 100. Communicator 301a may also receive history information from server 100. Communicator 301a may transmit food consumption information indicating the user's consumed food entered by the user to server 100. Communicator 301a may transmit a request for one or more first food consumption logs and receive food consumption log information indicating the one or more first food consumption logs. The one or more first food consumption logs are stored in storage 106 of server 100. The one or more first food consumption logs contain one or more instances of food consumption information regularly transmitted from portable terminal 300a. The one or more first food consumption logs are associated with user IDs and managed on a per user basis.

Input receiver 302a is an input interface (IF) that accepts inputs from the first user. Input receiver 302 may accept the selection of a dish for a meal that has been consumed (or is to be consumed) from a plurality of dish candidates. For example, the above selection may be made based on an input from the first user, made via the touch panel included in portable terminal 300a, of a dish among one or more dish candidates included in the user interface (UI) displayed on display 304a.

When communicator 301a receives support information or behavior information, controller 303a displays presentation information based on the received support information or behavior information on display 304a.

Display 304a is a display that displays images and UI. Display 304a displays images and UI generated by controller 303a. Display 304a is one example of the presenter that displays presentation information.

### Specific Example of Portable Terminal

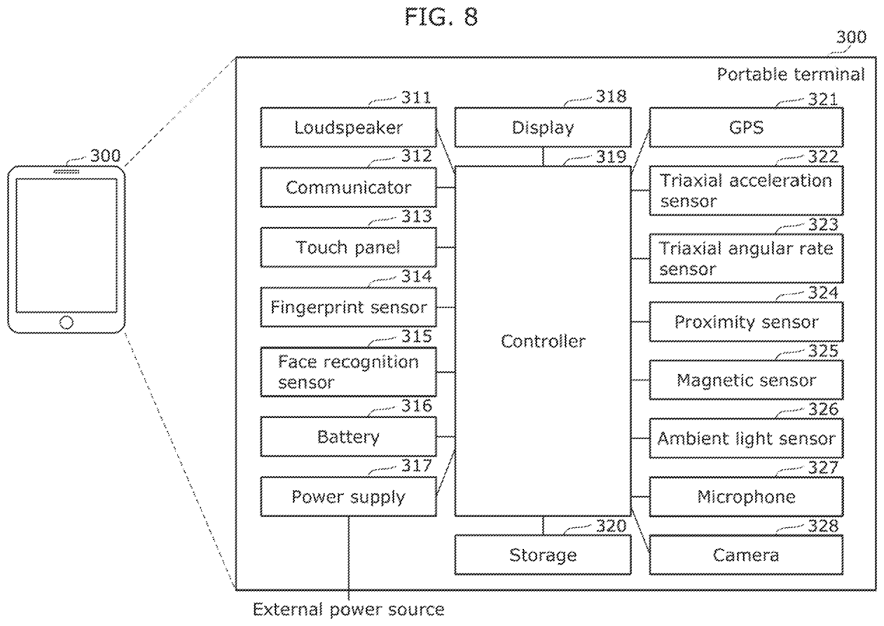

FIG. 8 illustrates the configuration of the portable terminal in detail. FIG. 8 schematically illustrates the block configuration of a smartphone as one example of the portable terminal.

As illustrated in FIG. 8, portable terminal 300 includes loudspeaker 311, communicator 312, touch panel 313, fingerprint sensor 314, face recognition sensor 315, battery 316, power supply 317, display 318, controller 319, storage 320, GPS 321, triaxial acceleration sensor 322, triaxial angular rate sensor 323, proximity sensor 324, magnetic sensor 325, ambient light sensor 326, microphone 327, and camera 328 are included.

Controller 319 is capable of integrally controlling portable terminal 300 and includes a central processing unit (CPU) and a memory element (such as SRAM) (not illustrated). Display 318 is a display that displays text and graphics based on information received from controller 319. Storage 320 stores the operating system (OS) to be read and executed by controller 319, various application programs (generally referred to as smartphone apps), and various data used by various programs. Storage 320 includes a nonvolatile storage area.

Communicator 312 implements wireless communication functions such as long term evolution (LTE) and 5G, and is wirelessly connected to a base station of a cellular phone communication network (not illustrated) and connected to the internet via the base station. Communicator 312 may implement wireless communication functions via Bluetooth or wireless LAN (for example, Wi-Fi). Communicator 312 may be connected to the internet via Bluetooth (registered trademark) or wireless LAN (for example, Wi-Fi (registered trademark)) through other communication devices.

Touch panel 313 accepts input from the user to the UI displayed on display 318 and transmits signals based on the input (for example, which part of the screen is being touched and with what degree of pressure) to controller 319. GPS 321 measures the geographical position of portable terminal 300. Triaxial acceleration sensor 322 sets the X-, Y-, and Z-axes for portable terminal 300, and measures acceleration in each axis. Triaxial angular rate sensor 323 measures angular velocity in the direction of rotation for each axis. Proximity sensor 324 detects objects in close proximity (for example, a face approaching the smartphone). Magnetic sensor 325 detects the geomagnetic field and indicates the direction. Ambient light sensor 326 detects the ambient brightness of portable terminal 300. Microphone 327 collects ambient sounds and speech. Camera 328 captures images from the front of portable terminal 300, where display 318 is located, or from the rear on the opposite side. Loudspeaker 311 emits sound based on audio signals generated by controller 319. Fingerprint sensor 314 is used for user authentication, etc., and detects a person's fingerprint. Face recognition sensor 315 authenticates a person's face. Face recognition sensor 315 may be realized by combining an infrared camera, a projection illuminator, a dot projector, etc., with a group of sensors (a proximity sensor, a camera, and an ambient light sensor).

Power supply 317 receives a supply of power from an external power source. Battery 316 stores the external power received by power supply 317 or supplies the stored power to portable terminal 300 for operating.

### Operations

Next, a first example of operations performed by lifestyle improvement system 10 for notifying support information will be described.

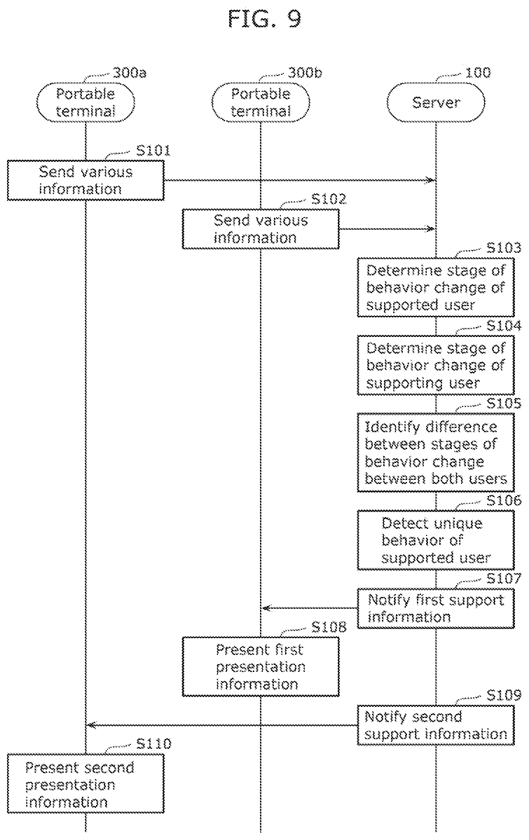

FIG. 9 is a sequence diagram illustrating a first example of operations performed by a lifestyle improvement system for notifying support information.

Portable terminal 300a sends various information to server 100 via network 400 (S101). The various information includes the first user's food consumption information and app information generated by the first user operating portable terminal 300a. The various information may include the first user's biometric information.

Portable terminal 300b sends various information to server 100 via network 400 (S102). The various information includes app information generated by the second user operating portable terminal 300b.

With steps S101 and S102, server 100 receives various information from portable terminals 300a and 300b and stores the various information in storage 106. The order of steps S101 and S102 may be reversed.

Next, server 100 determines the stage of behavior change of the supported user (S103). Server 100 evaluates the stage of behavior change of the first user based on the food consumption information, app information, and biometric information obtained from portable terminal 300a, and takes the evaluation result as the first stage of behavior change.

Next, server 100 determines the stage of behavior change of the supporting user (S104). Server 100 evaluates the stage of behavior change of the second user based on the app information obtained from portable terminal 300b, and takes the evaluation result as the second stage of behavior change.

The order of steps S103 and S104 may be reversed. Steps S103 and S104 may be performed at portable terminal 300a and portable terminal 300b, respectively, in which case portable terminal 300a transmits the determined first stage of behavior change to server 100 and portable terminal 300b transmits the determined second stage of behavior change to server 100. In this way, server 100 may obtain the first and second stages of behavior change from portable terminals 300a and 300b.

Next, server 100 compares the first stage of behavior change with the second stage of behavior change to identify a difference between the first stage of behavior change and the second stage of behavior change (S105).

Next, server 100 detects unique behavior of the supported user (S106). Unique behavior is behavior by the supported user that influences the transition of the supported user's stage of behavior change to a higher stage. Influencing a transition to a higher stage may be identified in accordance with the method of determining the stage of behavior change.

Next, if comparer 102 determines that there is a difference, server 100 notifies a low-evaluated user, which is a user whose stage of behavior change is evaluated low among the first user and the second user, with first support information corresponding to the stage of behavior change of the low-evaluated user and to the difference (S107). If the second stage of behavior change is lower than the first stage of behavior change, server 100 notifies the first support information to the second user's portable terminal 300b, as illustrated in FIG. 9. Conversely, if the first stage of behavior change is lower than the second stage of behavior change, server 100 notifies the first support information to the first user's portable terminal 300a.

Portable terminal 300b receives the first support information and presents first presentation information based on the received first support information (S108). Note that when portable terminal 300a receives the first support information, portable terminal 300a presents the first presentation information based on the received first support information.

Next, if it is determined that there is a difference, server 100 further notifies a high-evaluated user, which is a user whose stage of behavior change is evaluated high among the first user and the second user, with second support information corresponding to the stage of behavior change of the high-evaluated user and to the difference (S109). If the second stage of behavior change is lower than the first stage of behavior change, server 100 notifies the second support information to the first user's portable terminal 300a, as illustrated in FIG. 9. Conversely, if the first stage of behavior change is lower than the second stage of behavior change, server 100 notifies the second support information to the second user's portable terminal 300b.

Portable terminal 300a receives the second support information and presents second presentation information based on the received second support information (S110). Note that when portable terminal 300b receives the second support information, portable terminal 300b presents presentation information based on the received second support information.

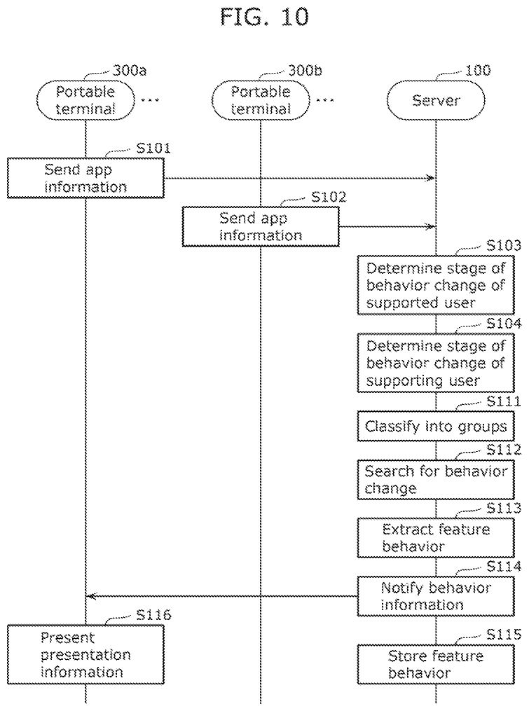

FIG. 10 is a sequence diagram illustrating a second example of operations performed by a lifestyle improvement system for notifying support information. In this sequence diagram, steps S101 through S104 are the same as in FIG. 9, so description will mainly focus on the different steps S111 through S115.

After step S104, server 100 classifies a plurality of users including a plurality of supported users and a plurality of supporting users into a plurality of groups according to the plurality of stages of behavior change obtained by evaluating each of the plurality of users, such that users evaluated to be in similar stages of behavior change are classified into the same group (S111). The groups may be classified by supported user or by supporting user. Groups may be further classified by attributes of the supported user. Attributes of the supported user may be the type of disease of the supported user, a classification based on biometric information of the supported user, a classification based on basic information (age, gender, height, weight) of the supported user, etc. Groups may be further classified by attributes of the supporting user. Attributes of the supporting user may be the type of disease of the supported user that the supporting user is supporting, a classification based on biometric information of the supporting user, a classification based on the supporting skills of the supporting user (for example, cooking skills).

Next, server 100 checks for a shift in behavior change from the food consumption information, app information, and biometric information subject to evaluation of the first and second user's stages of behavior change, and determines (searches for) the presence of a behavior change (S112). A shift in behavior change based on food consumption information includes, for example, a change in the content of consumed food (changes in estimated energy and salt intake), a change in meal times (regularity of meal times), a change in the content and frequency of eating/taking out, and a change in the amount and frequency of snacking. A shift in behavior change based on app information includes, for example, a change in frequency of opening the health management app, a change in time spent viewing health management app content, a change in time spent viewing recipes, a change in actually cooking recipes, and a change in frequency of using external services (food delivery, inquiries to experts via chat, etc.). A shift in behavior change based on biometric information includes, for example, a change in the number of times biometric information (blood pressure, weight, etc.) is recorded and viewed, and a change in the number of times exercise information is recorded and viewed (number of steps walked, etc.).

Next, server 100 extracts a feature behavior (S113). Server 100 extracts the trigger of the behavior that led to the behavior change of the first or second user, as well as the feature behavior, which is the behavior that was effective in bringing about the behavior change. A feature behavior based on food consumption information is, for example, the continued registration of photographs and manual input of meal content. This allows us to assume that the user is aware of the changes in meal content (i.e., the amount of energy or salt intake estimated from the photograph). A feature behavior based on food consumption information is that the content and frequency of eating/taking out are changing, and as a result, is a behavior when it can be inferred that there is a change in the first user's biometric information. A feature behavior based on food consumption information is a change (decrease) in the amount of snacking, a change in snack content (for example, from snack food to fruit), and a change (decrease) in the frequency of snacking. A feature behavior based on food consumption information is the frequency of choosing low-sodium meal kits, the frequency of choosing low-sodium boxed lunches, and a change (increase) in the frequency of cooking low-sodium recipes. A feature behavior based on app information is a change (increase) in the frequency of opening the health management app and a change (increase) in the time spent viewing the app. A feature behavior based on app information is a change (increase) in recipe viewing time (increase in time) and a change in cooking meals (i.e., an increase in the number of times a meal is cooked). A feature behavior based on app information is a change (increase) in the frequency of use of external services. A feature behavior based on biometric information is a change (increase) in the number of times biometric information is recorded, a change (increase) in the number of times exercise information is recorded, and continued regular recording.

Next, server 100 may notify one or more users who currently belong to the same group to which the user belonged at the time of the feature behavior (S114) with the behavior information indicating the extracted feature behavior.

Next, server 100 stores the behavior information indicating the extracted feature behavior in storage 106 for each group to which the user belonged at the time of the feature behavior (S115).

The one or more portable terminals 300a possessed by one or more users belonging to the group that was the target of the notification receive the behavior information and present presentation information (behavior) based on the received behavior information (S116).

### Operations Related to Input of Food Consumption Log Entries

Next, one example of operations related to input of food consumption log entries in lifestyle improvement system 10 will be described.

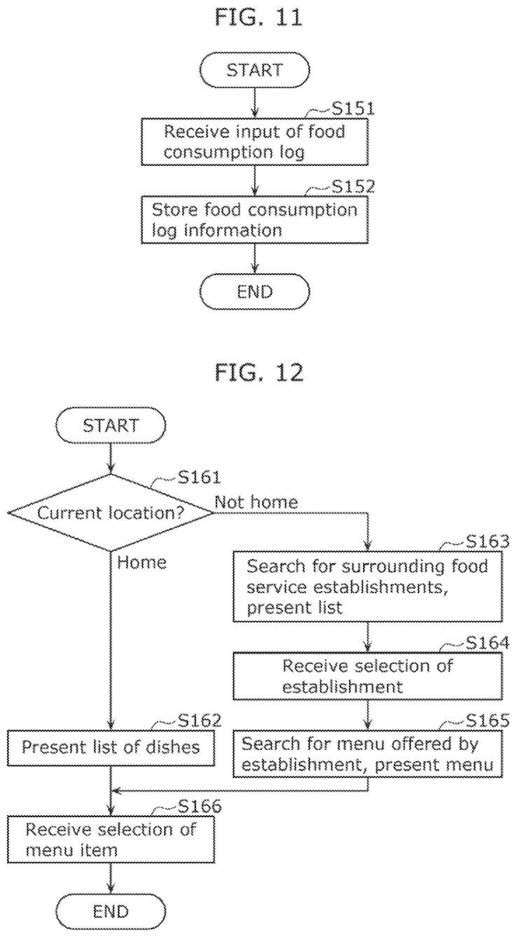

FIG. 11 is a flowchart illustrating one example of operations related to input of food consumption log entries in the lifestyle improvement system.

Portable terminal 300 receives an input of a food consumption log (S151). A specific example of step S151 will be given later.

Next, portable terminal 300 generates food consumption log information based on the received input and stores the generated food consumption log information (S152). Note that the food consumption log information may be transmitted to server 100 and stored in server 100.

### First Example

FIG. 12 is a flowchart illustrating a first example of a process of inputting a food consumption log.

When the user uses an app on portable terminal 300 to input a food consumption log, portable terminal 300 obtains the current location using GPS or other some means, compares the obtained current location with the location information of the user's home, and determines whether the current location is the user's home or not (S161).

If portable terminal 300 determines that the current location is the user's home (“home” in S161), it estimates that the meal being entered by the user was prepared in the home and presents the user with a list of dishes stored in the app or in server 100 connected via the internet (S162). In step S166 after this, a food consumption log is input by portable terminal 300 receiving the user's selection of an eaten (or to be eaten) dish. Other possible methods for inputting food consumption log entries include taking pictures or extraction from the browsing history of cooking recipes.

If portable terminal 300 determines that the current location is outside the home (“not home” in S161), it estimates that the meal being entered by the user is from a food service establishment, and searches for a restaurant or other food service establishment near the current location based on location information. Portable terminal 300 then presents the user with a list of potential food service establishments (S163). When presenting this list of food service establishment, portable terminal 300 may highlight establishments that the user has patronized to make the selection easier for the user. Next, portable terminal 300 receives the user's selection of a food service establishment (S164).

Next, portable terminal 300 searches for the menu offered by the selected food service establishment and, if the menu is disclosed, presents the menu to the user (S165). In step S166 after this, a food consumption log is input by portable terminal 300 receiving the user's selection of an eaten (or to be eaten) menu item. If the food service establishment also discloses nutritional information such as salt content in conjunction with the menu information, portable terminal 300 may obtain the nutritional information.

This makes it easy to identify the food service establishment where the meal was eaten and the menu of the meals served there, thereby reducing the time and effort required to input a food consumption log. At the same time, if nutritional information is disclosed, the accuracy of, for example, the amount of salt intake can be expected to improve.

It is known that meals from food service establishments tend to contain more salt than those prepared at home, even if the dish is the same. The reason is that a lot of salt tends to be used to make meals taste better and improve the preservation of food. For example, it is known that nikujaga consumed at food service establishments has a higher salt content than nikujaga prepared at home.

Therefore, if a meal is estimated to be from a food service establishment via the processes described above, the estimated salt intake corresponding to the input food consumption log may be calculated at a higher rate than normal (home-cooked meals). This makes it possible to take into account a higher-than-expected salt intake due to a meal being from a food service establishment, and inhibits deviations from the target salt intake.

### Second Example

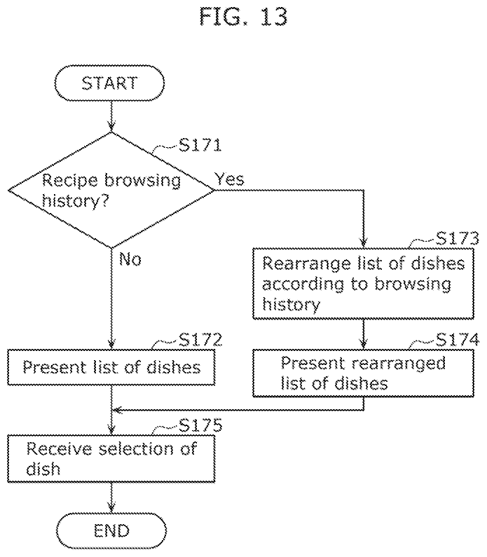

FIG. 13 is a flowchart illustrating a second example of a process of inputting a food consumption log.

The app that manages the food consumption logs on portable terminal 300 may have a function for searching for cooking recipes.

When the user uses the app on portable terminal 300 to input a food consumption log, portable terminal 300 determines whether there is a browsing history of cooking recipes (S171).

If portable terminal 300 determines that there is no browsing history of cooking recipes (No in S171), it presents the user with a list of dishes stored in the app or in server 100 connected via the internet (S172). In step S175 after this, a food consumption log is input by portable terminal 300 receiving the user's selection of an eaten (or to be eaten) dish. Other possible methods for inputting food consumption log entries include taking pictures.

If portable terminal 300 determines that there is a browsing history of cooking recipes (Yes in S171), it rearranges the list of dishes stored in the app or in server 100 connected via the internet according to the browsing history (S173). Specifically, portable terminal 300 rearranges the list of dishes so that dishes corresponding to the cooking recipes included in the browsing history are at the top of the list. When a cooking recipe is browsed, portable terminal 300 may include the dishes of the meal of the cooking recipe itself in the browsing history and present it to the user in the list of dishes.

Portable terminal 300 then presents the rearranged list of dishes to the user (S174). In step S175 after this, a food consumption log is input by portable terminal 300 receiving the user's selection of an eaten (or to be eaten) dish. Other possible methods for inputting food consumption log entries include taking pictures.

With this, dishes that are more likely to have been cooked will appear higher on the list, making it easier for users to select dishes they have eaten.

### Third Example

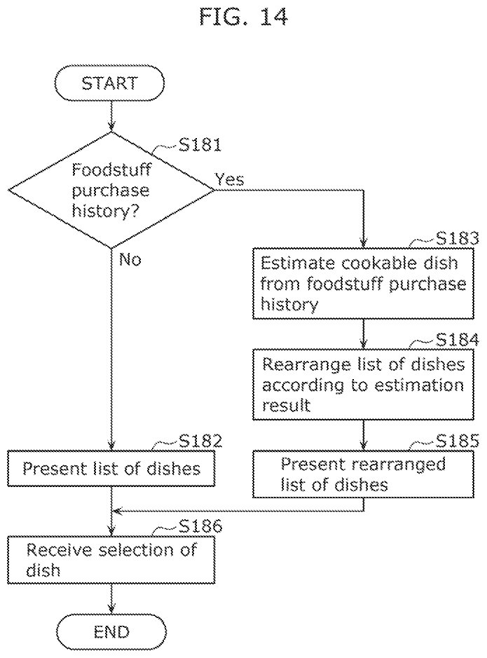

FIG. 14 is a flowchart illustrating a third example of a process of inputting a food consumption log.

The app that manages the food consumption logs on portable terminal 300 may have a function for storing or searching a purchase history of foodstuffs purchased from stores and the like.

When the user uses the app on portable terminal 300 to input a food consumption log, portable terminal 300 determines whether there is a purchase history of foodstuffs (S181).

If portable terminal 300 determines that there is no purchase history of foodstuffs (No in S181), it presents the user with a list of dishes stored in the app or in server 100 connected via the internet (S182). In step S186 after this, a food consumption log is input by portable terminal 300 receiving the user's selection of an eaten (or to be eaten) dish. Other possible methods for inputting food consumption log entries include taking pictures or extraction from the browsing history of cooking recipes.

If portable terminal 300 determines that there is a purchase history of foodstuffs (Yes in S181), it estimates a dish that could be cooked from the purchase history of foodstuffs (S183).

Next, portable terminal 300 rearranges the list of dishes stored in the app or in server 100 connected via the internet according to the result of the estimation (S184). Specifically, portable terminal 300 rearranges the list of dishes so that dishes corresponding to result of the estimation are at the top of the list.

Portable terminal 300 then presents the rearranged list of dishes to the user (S185). In step S186 after this, a food consumption log is input by portable terminal 300 receiving the user's selection of an eaten (or to be eaten) dish. Other possible methods for inputting food consumption log entries include taking pictures or extraction from the browsing history of cooking recipes.

With this, dishes that are more likely to have been cooked will appear higher on the list, making it easier for users to select dishes they have eaten.

### Fourth Example

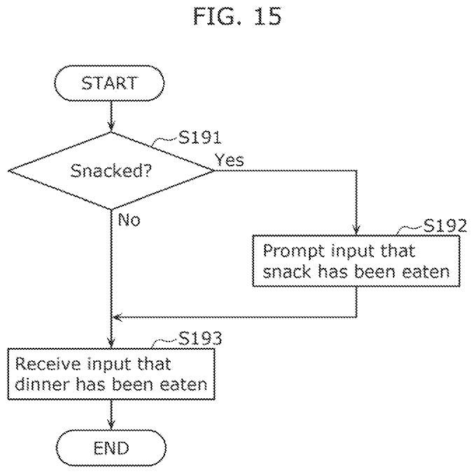

FIG. 15 is a flowchart illustrating a fourth example of a process of inputting a food consumption log.

When the user uses the app on portable terminal 300 to input a food consumption log, portable terminal 300 determines whether the user has eaten a snack (S191). More specifically, during the time of day at which dinner is typically eaten (for example, from 17:00 to 22:00), if no food consumption log has been input since the input for lunch, portable terminal 300 presents a UI that asks the user if they have eaten any snacks or other the like between lunch and dinner (i.e., if they have snacked between meals). Portable terminal 300 determines that the user has eaten a snack when an input indicating such is made, and determines that the user has not eaten a snack an input indicating such is made.

If portable terminal 300 determines that the user has eaten a snack (Yes in S191), it prompts the user to input that they have eaten a snack (S192).

If portable terminal 300 determines that the user has not eaten a snack (No in S191), or after step S192, it receives an input from the user that they have eaten dinner (S193).

This allows portable terminal 300 to receive an input that a snack was eaten along with an input that dinner has been eaten in cases in which the user ate a snack before dinner.

Portable terminal 300 may similarly confirm with the user that they did not eat a snack after dinner the previous night upon the user inputting a food consumption log for breakfast, and confirm with the user that they did not eat a snack after breakfast upon the user inputting a food consumption log for lunch.

Regarding snacks after dinner, portable terminal 300 may perform the detection before the user goes to bed and confirm with the user that they did not eat a snack after dinner.

Portable terminal 300 may confirm with the user that there are no omissions in entries in the food consumption log for the three meals (breakfast, lunch, and dinner) when checking before the user goes to bed.

When portable terminal 300 receives an input of a food consumption log for dinner from the user via the app that manages the food consumption logs, if the user has a history of meal delivery service usage in other apps, portable terminal 300 may obtain the history from that smartphone app and display it as an option for the input of the food consumption log.

The above acts as a prevention against forgetting to input food consumption log entries for snacks, which are easy to forget and can be a burden, and thus reduces the time and effort required for inputting food consumption log entries.

### Advantageous Effects, etc.

Lifestyle improvement system 10 according to the present embodiment includes obtainer 101, comparer 102, and notifier 103. Obtainer 101 obtains a first stage of behavior change of a first user who receives support for lifestyle improvement and a second stage of behavior change of a second user who supports the lifestyle improvement of the first user. Comparer 102 compares the first stage of behavior change with the second stage of behavior change to identify a difference between the first stage of behavior change and the second stage of behavior change. When the comparer identifies the difference, notifier 103 notifies a low-evaluated user with first support information. The low-evaluated user is a user whose stage of behavior change is evaluated low among the first user and the second user. The first support information corresponds to the stage of behavior change of the low-evaluated user and to the difference. The first support information is for raising the stage of behavior change of the low-evaluated user.

With this, if there is a difference between the first and second stages of behavior change, the low-evaluated user is notified with first support information corresponding to the lower stage of behavior change and to the difference, and thus the stage of behavior change of the low-evaluated user can be effectively raised.

Lifestyle improvement system 10 according to the present embodiment further includes storage 106. Storage 106 stores a plurality of instances of the first support information. Each instance of the first support information is associated with a plurality of combinations of a plurality of stages of behavior change in which the first user is evaluatable and a plurality of stages of behavior change in which the second user is evaluatable. Notifier 103 determines, as the first support information to notify the low-evaluated user with, first support information that is associated with a combination of the first stage of behavior change and the second stage of behavior change from among the plurality of instances of the first support information, and notifies the low-evaluated user with the first support information determined.

This makes it possible to determine first support information that corresponds to the lower stage of behavior change and to the difference and is suited to the low-evaluated user.

In lifestyle improvement system 10 according to the present embodiment, when comparer 102 identifies the difference, notifier 103 further notifies a high-evaluated user with second support information. The high-evaluated user is a user whose stage of behavior change is evaluated high among the first user and the second user. The second support information corresponds to the stage of behavior change of the high-evaluated user and to the difference. The second support information is for maintaining or raising the stage of behavior change of the high-evaluated user.

With this, if there is a difference between the first and second stages of behavior change, the high-evaluated user is further notified with second support information corresponding to the higher stage of behavior change and to the difference, and thus the stage of behavior change of the high-evaluated user can be effectively raised.

Lifestyle improvement system 10 according to the present embodiment further includes storage 106. Storage 106 stores a plurality of instances of the first support information and a plurality of instances of the second support information. Each instance of the first support information is associated with a plurality of combinations of a plurality of stages of behavior change in which the first user is evaluatable and a plurality of stages of behavior change in which the second user is evaluatable. Each instance of the second support information is associated with a plurality of combinations of a plurality of stages of behavior change in which the first user is evaluatable and a plurality of stages of behavior change in which the second user is evaluatable. Notifier 103 determines, as the first support information to notify the low-evaluated user with, first support information that is associated with a combination of the first stage of behavior change and the second stage of behavior change from among the plurality of instances of the first support information, and notifies the low-evaluated user with the first support information determined. Notifier 103 determines, as the second support information to notify the high-evaluated user with, second support information that is associated with a combination of the first stage of behavior change and the second stage of behavior change from among the plurality of instances of the second support information, and notifies the high-evaluated user with the second support information determined.

This makes it possible to determine first support information that corresponds to the lower stage of behavior change and to the difference and is suited to the low-evaluated user as well as second support information that corresponds to the higher stage of behavior change and to the difference and is suited to the high-evaluated user.

In lifestyle improvement system 10 according to the present embodiment, the first stage of behavior change is determined at predetermined intervals according to at least one of: a record of daily activity of the first user; a viewing history on a first terminal (portable terminal 300a) possessed by the first user of an app for managing a health condition of the first user; biometric information of the first user; or an amount of exercise by the first user. The second stage of behavior change is determined at the predetermined intervals according to: a viewing history on a second terminal (portable terminal 300b) possessed by the second user of an app for managing the health condition of the first user; and a cooking skill level of the second user.

With this, since a first stage of behavior change appropriate for the behavior and state of the first user and a second stage of behavior change appropriate for the behavior and state of the second user are determined, it is possible to determine first support information that can effectively raise the stage of behavior change of the low-evaluated user.

In lifestyle improvement system 10 according to the present embodiment, the stage of behavior change is an indicator of a degree of willingness to change daily behavior to transition from a first state to a second state improved relative to the first state.

This makes it possible to determine a stage of behavior change according to the degree of the user's willingness to change their daily behavior.

In lifestyle improvement system 10 according to the present embodiment, the first support information includes at least one of advice, encouragement, or behavior information indicating a lifestyle improvement behavior of the high-evaluated user, each of which is for raising the stage of behavior change of the low-evaluated user.

This makes it possible to effectively raise the stage of behavior change of the low-evaluated user.

Lifestyle improvement system 10 according to the present embodiment includes obtainer 101, classifier 104, extractor 105, and notifier 103. Obtainer 101 obtains a plurality of stages of behavior change by evaluating the stage of behavior change of each of a plurality of users. Classifier 104 classifies the plurality of users into a plurality of groups according to the plurality of stages of behavior change such that users evaluated to be in similar stages of behavior change are classified into the same group. Extractor 105 obtains one or more first behaviors of each of one or more users belonging to a first group among the plurality of groups, and extracts a second behavior that affects the transition to a higher stage of behavior change from among the obtained one or more first behaviors. Among one or more user, notifier 103 notifies one or more users currently belonging to the first group with the second behavior.

With this, since one or more third users currently belonging to the first group are notified with the second behavior that affects the transition to a higher stage of behavior change among one or more first behaviors of each of one or more second users belonging to the first group, it is possible to raise the stage of behavior change of the third user to a higher stage of behavior change.

Moreover, in lifestyle improvement system 10 according to the present embodiment, the one or more first behaviors are behaviors of one or more third users.

With this, since the second behavior is extracted from among the behaviors of one or more third users currently belonging to the first group, it is possible to notify behavior information indicating the behavior of a user classified in the same group as the one or more third users subject to be notified. Accordingly, there is potential to more effectively raise the stage of behavior change of the one or more third users to a higher stage of behavior change.

Moreover, in lifestyle improvement system 10 according to the present embodiment, the one or more first behaviors are behaviors that influenced one or more fourth users currently belonging to a second group evaluated at a higher stage of behavior change relative to the first group to transition from the first group to the second group.

With this, since a behavior of one or more fourth users who belonged to the first group which affected their transition from the first group to the second group is extracted as the second behavior, it is possible to extract a behavior having a proven track record of affecting group transitions as the second behavior. In other words, there is potential to more effectively raise the stage of behavior change of the one or more third users to a higher stage of behavior change.

In lifestyle improvement system 10 according to the present embodiment, the plurality of first users include a plurality of supported users who receive support for lifestyle improvement and a plurality of supporting users who support the lifestyle improvement of the plurality of supported users.

In lifestyle improvement system 10 according to the present embodiment, the stage of behavior change of the supported user is determined at predetermined intervals according to at least one of: a record of daily activity of the supported user; a viewing history on a first terminal (portable terminal 300a) possessed by the supported user of an app for managing a health condition of the supported user; biometric information of the supported user; or an amount of exercise by the supported user. The stage of behavior change of the supporting user is determined at predetermined intervals according to a viewing history on a second terminal (portable terminal 300b) possessed by the supporting user of an app for managing a health condition of the supporting user, and a supporting skill of the supporting user.

With this, since a first stage of behavior change appropriate for the behavior and state of the supported user and a second stage of behavior change appropriate for the behavior and state of the supporting user are determined, it is possible to notify one or more users currently belonging to the first group of the second behavior that can effectively raise the stage of behavior change of the low-evaluated user.

In lifestyle improvement system 10 according to the present embodiment, the stage of behavior change is an indicator of a degree of willingness to change daily behavior to transition from a first state to a second state improved relative to the first state.

This makes it possible to determine a stage of behavior change according to the degree of the user's willingness to change their daily behavior.

### Variations

In lifestyle improvement system 10 according to the above embodiment, at least one of portable terminal 300a or portable terminal 300b may include all or part of the functions of obtainer 101, comparer 102, notifier 103, classifier 104, extractor 105, and storage 106 included in server 100. In lifestyle improvement system 10, server 100 may include all or part of the functions of controller 303a included in portable terminal 300a and controller 303b included in portable terminal 300b.

### Other Comments

Each of the elements in each of the above-described embodiments may be implemented as dedicated hardware, or may be realized by executing a software program suitable for the element. Each of the elements may be realized by way of a program executing unit, such as a CPU or a processor, reading and executing the software program recorded on a recording medium such as a hard disk or a semiconductor memory.

As described above, embodiments have been described as examples of techniques according to the present disclosure. The accompanying drawings and detailed description are provided for this purpose.

Therefore, elements described in the accompanying drawings and the detailed description include, in addition to elements essential to overcoming the technical problem, elements that are not essential to overcoming the technical problem but are included in order to exemplify the techniques described above. As such, description of these non-essential elements in the accompanying drawings and the detailed description should not be taken to mean that these non-essential elements are essential.

The above embodiments are for providing examples of the techniques according to the present disclosure, and thus various modifications, substitutions, additions, and omissions are possible within the scope of the claims and equivalents thereof.

### INDUSTRIAL APPLICABILITY

The present disclosure is applicable to a lifestyle improvement system, etc., that can effectively raise the stage of behavior change of a user who receives support for lifestyle improvement and a user who supports the lifestyle improvement of the previously mentioned user.

1. A lifestyle improvement system comprising:
an obtainer that obtains a plurality of first stages of behavior change by evaluating a stage of behavior change of each of a plurality of first users;
a classifier that classifies the plurality of first users into a plurality of groups according to the plurality of first stages of behavior change, grouping users evaluated to be in similar stages of behavior change together;
an extractor that obtains one or more first behaviors of each of one or more second users belonging to a first group among the plurality of groups, and extracts, from among the one or more first behaviors obtained, a second behavior that affects transition to a higher stage of behavior change; and
a notifier that notifies one or more third users currently belonging to the first group among the one or more second users with behavior information indicating the second behavior.

2. The lifestyle improvement system according to claim 1, wherein
the one or more first behaviors are behaviors of the one or more third users.

3. The lifestyle improvement system according to claim 1, wherein
the one or more first behaviors are behaviors that influenced one or more fourth users currently belonging to a second group evaluated at a higher stage of behavior change relative to the first group to transition from the first group to the second group.

4. The lifestyle improvement system according to claim 1, wherein
the plurality of first users include a plurality of supported users who receive support for lifestyle improvement and a plurality of supporting users who support the lifestyle improvement of the plurality of supported users.

5. The lifestyle improvement system according to claim 4, wherein
the first stage of behavior change of each of the plurality of supported users is determined at predetermined intervals according to at least one of: a record of daily activity of the supported user;
a viewing history on a first terminal possessed by the supported user of an app for managing a health condition of the supported user; biometric information of the supported user; or an amount of exercise by the supported user, and
the first stage of behavior change of each of the plurality of supporting users is determined at predetermined intervals according to a viewing history on a second terminal possessed by the supporting user of an app for managing a health condition of the supporting user, and a supporting skill of the supporting user.

6. The lifestyle improvement system according to claim 1, wherein
the stage of behavior change is an indicator of a degree of willingness to change daily behavior to transition from a first state to a second state improved relative to the first state.

7. A portable terminal connected to a server over a network, the server including:
an obtainer that obtains a plurality of first stages of behavior change by evaluating a stage of behavior change of each of a plurality of first users;
a classifier that classifies the plurality of first users into a plurality of groups according to the plurality of first stages of behavior change, grouping users evaluated to be in similar stages of behavior change together;
an extractor that obtains one or more first behaviors of each of one or more second users belonging to a first group among the plurality of groups, and extracts, from among the one or more first behaviors obtained, a second behavior that affects transition to a higher stage of behavior change; and
a notifier that notifies one or more third users currently belonging to the first group among the one or more second users with behavior information indicating the second behavior,
the portable terminal comprising:
a receiver that receives the behavior information; and
a presenter that presents the second behavior based on the behavior information received.

8. A control method of a lifestyle improvement system including one or more processors and a presenter, the control method comprising, executed by the one or more processors:
obtaining a plurality of first stages of behavior change by evaluating a stage of behavior change of each of a plurality of first users;
obtaining one or more first behaviors of each of one or more second users belonging to a first group among a plurality of groups into which the plurality of first users have been classified, the plurality of first users being classified according to the plurality of first stages of behavior change, grouping users evaluated to be in similar stages of behavior change together; and
notifying one or more third users currently belonging to the first group among the one or more second users with behavior information indicating a second behavior that affects transition to a higher stage of behavior change from among the one or more first behaviors obtained.

9. A non-transitory computer-readable recording medium having recorded thereon a program for causing a computer to execute the control method according to claim 8.
